(19) United States
(12) Patent Application Publication
(10) Pub. No.: US 2004/0024296 A1
(43) Pub. Date: Feb. 5, 2004

(54) System, method and computer program product for screening a spectral image

(76) Inventors: Eric P. Krotkov, Wynnewood, PA (US); Yongping Wang, Philadelphia, PA (US); Zhang Zhong, Herndon, VA (US); Robert M. Danen, San Jose, CA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/362,643

(22) PCT Filed: Aug. 27, 2001
(86) PCT No.: PCT/US01/26653

(51) Int. Cl. A61B 5/00

(52) U.S. Cl. 600/322

(57) ABSTRACT

A system, method and computer program product is provided for screening and analyzing spectral images of a microcirculatory system to measure blood characteristics, such as hemoglobin concentration. In an embodiment, multi-stage screening is performed on each input image to determine whether the image is suitable for hemoglobin measurement and prediction. During the screening process the following quantities are measured: image intensity, image background intensity variation, image focus condition, image motion blur condition, number of vessels in the image with certain range of diameters, average vessel edge contrast, number of bubble segments in the image and the similarity between two images. The multi-stage screening cycle includes intensity screening, DWT decomposition, focus measurement, motion blur measurement, vessel and bubble detection, and repetition detection. During the screening stages, the image is classified into one of three categories: excellent, mediocre and poor. Only the images with an excellent rating is used for further Hb estimation. But both excellent and mediocre images would launch an autofocusing process provided by focus measurement. In another embodiment, a Fast Fourier transform is used to develop a spectral ratio to detect excellent images. The present invention provides real time hemoglobin prediction on the subject under investigation.

### BACKGROUND OF THE INVENTION

1. Field of the Invention

The present invention relates generally to reflected light analysis. More particularly, the invention relates to the use of reflected spectral imaging to analyze visualizable components of a fluid flowing in a tubular system. Still more particularly, the invention relates to screening reflected spectral images to analyze visualizable components of fluids in a vascular system.

2. Related Art

Widely accepted medical school doctrine teaches that the complete blood count including the white blood cell differential (CBC+Diff) is one of the best tests to assess a patient's overall health. With it, a physician can detect or diagnose anemia, infection, blood loss, acute and chronic diseases, allergies, and other conditions. CBC+Diff analyses provide comprehensive information on constituents in blood, including the number of red cells, the hematocrit, the hemoglobin concentration, and indices that portray the size, shape, and oxygen-carrying characteristics of the entire red blood cell (RBC) population. The CBC+Diff also includes the number and types of white blood cells and the number of platelets. The CBC+Diff is one of the most frequently requested diagnostic tests with about two billion done in the United States per year.

A conventional CBC+Diff test is done in an “invasive” manner in which a sample of venous blood is drawn from a patient through a needle, and submitted to a laboratory for analysis. For example, a phlebotomist (an individual specially trained in drawing blood) collects a sample of venous blood into a tube containing an anticoagulant to prevent the blood from clotting. The sample is then sent to a hematology laboratory to be processed, typically on automated, multiparameter analytical instruments, such as those manufactured by Coulter Diagnostics of Miami, Fla. The CBC+Diff test results are returned to the requesting physician, typically on the next day.

In medical diagnosis it is often necessary to measure other types of blood components, such as non-cellular constituents present in the plasma component of blood. Such constituents can include, for example, blood gases and bilirubin. Bilirubin is a reddish to yellow pigment produced in the metabolic breakdown of hemoglobin and other proteins. Bilirubin is removed from the blood by the liver and is excreted from the body. However, the livers of newborn children, especially premature babies, cannot process bilirubin effectively.

The birth process often results in extensive bruising, resulting in blood escaping into the tissues where it is broken down metabolically. For this and other medical causes, bilirubin may accumulate in the blood stream. If bilirubin levels rise high enough, it begins to be deposited in other body tissues causing jaundice. Its first appearance is in the eye. At still higher levels, deposition begins in deeper tissues, including the brain, and can result in permanent brain damage.

The most common method for bilirubin analysis is through an in vitro process. In such an in vitro process, a blood sample is invasively drawn from the patient. The formed elements (red blood cells and other cells) are separated by centrifugation and the remaining fluid is reacted chemically and analyzed spectrophotometrically.

Invasive techniques, such as for conventional CBC+Diff tests and bilirubin analysis, pose particular problems for newborns because their circulatory system is not yet filly developed. Blood is typically drawn using a “heel stick” procedure wherein one or more punctures are made in the heel of the newborn, and blood is repeatedly squeezed out into a collecting tube. This procedure is traumatic even for an infant in good health. More importantly, this procedure poses the risk of having to do a blood transfusion because of the low total blood volume of the infant. The total blood volume of the newborn infant is 60-70 cc/kg body weight. Thus, the total blood volume of low birth weight infants (under 2500 grams) cared for in newborn intensive care units ranges from 45-175 cc. Because of their low blood volume and delay in production of red cells after birth, blood sampling from preterm infants and other sick infants frequently necessitates transfusions for these infants. Blood bank use for transfusion of infants in neonatal intensive care units is second only to the usage for cardiothoracic surgery. In addition to newborns, invasive techniques are also particularly stressful for, and/or difficult to carry out on, children, elderly patients, bum patients, and patients in special care units.

A hierarchical relationship exists between the laboratory findings and those obtained at the physical examination. The demarcation between the physical findings of the patient and the laboratory findings are, in general, the result of technical limitations. For instance, in the diagnosis of anemia (defined as low hemoglobin concentration), it is frequently necessary to quantify the hemoglobin concentration or the hematocrit in order to verify the observation of pallor. Pallor is the lack of the pink color of skin which frequently signals the absence or reduced concentration of the heavily red pigmented hemoglobin. However, there are some instances in which pallor may result from other causes, such as constriction of peripheral vessels, or being hidden by skin pigmentation. Because certain parts of the integument are less affected by these factors, clinicians have found that the pallor associated with anemia can more accurately be detected in the mucous membrane of the mouth, the conjunctivae, the lips, and the nail beds. A device which is able to rapidly and non-invasively quantitatively determine the hemoglobin concentration directly from an examination of one or more of the foregoing areas would eliminate the need to draw a venous blood sample to ascertain anemia. Such a device would also eliminate the delay in waiting for the laboratory results in the evaluation of the patient. Such a device also has the advantage of added patient comfort.

Soft tissue, such as mucosal membranes or unpigmented skin, do not absorb light in the visible and near-infrared, i.e., they do not absorb light in the spectral region where hemoglobin absorbs light. This allows the vascularization to be differentiated by spectral absorption from surrounding soft tissue background. However, the surface of soft tissue strongly reflects light and the soft tissue itself effectively scatters light after penetration of only 100 microns. Therefore, in vivo visualization of the circulation is difficult because of poor resolution, and generally impractical because of the complexities involved in compensating for multiple scattering and for specular reflection from the surface. Studies on the visualization of cells in the microcirculation consequently have been almost exclusively invasive, using a thin section (less than the distance for multiple scattering) of tissue containing the microcirculation, such as the mesentery, that can be observed by a microscope using light transmitted through the tissue section. Other studies have experimented with producing images of tissues from within the multiple scattering region by time gating (see, Yodh, A. and B. Chance, Physics Today, March, 1995, 34-40). However, the resolution of such images is limited because of the scattering of light, and the computations for the scattering factor are complex.

Spectrophotometry involves analysis based on the absorption or attenuation of electromagnetic radiation by matter at one or more wavelengths of light. The instruments used in this analysis are referred to as spectrophotometers. A simple spectrophotometer includes: a source of radiation, such as, e.g., a light bulb; a spectral selection means, such as a monochromator containing a prism or grating or colored filter; and one or more detectors, such as, e.g., photocells, which measure the amount of light transmitted and/or reflected by the sample in the selected spectral region.

In opaque samples, such as solids or highly absorbing solutions, the radiation reflected from the surface of the sample maybe measured and compared with the radiation reflected from a non-absorbing or white sample. If this reflectance intensity is plotted as a function of wavelength, it gives a reflectance spectrum. Reflectance spectra are commonly used in matching colors of dyed fabrics or painted surfaces. However, because of its limited range and inaccuracy, reflection spectrophotometry has been used primarily in qualitative rather than quantitative analysis. On the other hand, transmission spectrophotometry is conventionally used for quantitative analysis because Beer's law (inversely relating the logarithm of measured intensity linearly to concentration) can be used.

Reflective spectrophotometry is conventionally avoided for quantitative analysis because specularly reflected light from a surface limits the available contrast (black to white or signal to noise ratio), and, consequently, the measurement range and linearity. Because of surface effects, measurements are usually made at an angle to the surface. However, only for the special case of a Lambertian surface will the reflected intensity be independent of the angle of viewing. Light reflected from a Lambertian surface appears equally bright in all directions (cosine law). However, good Lambertian surfaces are difficult to obtain. Conventional reflection spectrophotometry presents an even more complicated relationship between reflected light intensity and concentration than exists for transmission spectrophotometry which follows Beer's law. Under the Kubelka-Munk theory applicable in reflection spectrophotometry, the intensity of reflected light can be related indirectly to concentration through the ratio of absorption to scattering.

Some imaging studies have been done in the reflected light of the microcirculation of the nail beds on patients with Raynauds, diabetes, and sickle cell disease. These studies were done to obtain experimental data regarding capillary density, capillary shape, and blood flow velocity, and were limited to gross physical measurements on capillaries. No spectral measurements, or individual cellular measurements, were made, and Doppler techniques were used to assess velocity. The non-invasive procedure employed in these studies could be applied to most patients, and in a comfortable manner.

One non-invasive device for in vivo analysis is disclosed in U.S. Pat. No. 4,998,533 to Winkelman. The Winkelman device uses image analysis and reflectance spectrophotometry to measure individual cell parameters such as cell size. Measurements are taken only within small vessels, such as capillaries where individual cells can be visualized. Because the Winkelman device takes measurements only in capillaries, measurements made by the Winkelman device will not accurately reflect measurements for larger vessels. This inaccuracy results from the constantly changing relationship of volume of cells to volume of blood in small capillaries resulting from the non-Newtonian viscosity characteristic of blood. Consequently, the Winkelman device is not capable of measuring the central or true hematocrit, or the total hemoglobin concentration, which depend upon the ratio of the volume of red blood cells to that of the whole blood in a large vessel such as a vein.

The Winkelman device measures the number of white blood cells relative to the number of red blood cells by counting individual cells as they flow through a micro-capillary. The Winkelman device depends upon accumulating a statistically reliable number of white blood cells in order to estimate the concentration. However, blood flowing through a micro-capillary will contain approximately 1000 red cells for every white cell, making this an impractical method. The Winkelman device does not provide any means by which platelets can be visualized and counted. Further, the Winkelman device does not provide any means by which the capillary plasma can be visualized, or the constituents of the capillary plasma quantified. The Winkelman device also does not provide a means by which abnormal constituents of blood, such as tumor cells, can be detected.

Another non-invasive device for in vivo analysis is disclosed in commonly assigned U.S. Pat. No. 5,983,120, issued Nov. 9, 1999, in the names of Warren Groner and Richard G. Nadeau, and entitled “Method and Apparatus for Reflected Imaging Analysis” (hereinafter referred to as “the '120 patent”), or in commonly assigned U.S. Pat. No. 6,104,939, issued Aug. 15, 2000, in the names of Warren Groner and Richard G. Nadeau, and entitled “Method and Apparatus for Reflected Emaging Analysis” (hereinafter referred to as “the '939 patent”). The disclosure of the '120 patent and the '939 patent are incorporated herein by reference as though set forth in its entirety. The device of the '120 patent or the '939 patent provides for complete non-invasive in vivo analysis of a vascular system. This device provides for high resolution visualization of blood cell components (red blood cells, white blood cells, and platelets), blood rheology, blood vessels, and vascularization throughout the vascular system. The device of the '120 patent or the '939 patent allows quantitative determinations to be made for blood cells, normal and abnormal contents of blood cells, as well as for normal and abnormal constituents of blood plasma.

The device of the '120 patent or the '939 patent captures a raw reflected image of a blood sample, and normalizes the image with respect to the background to form a corrected reflected image. An analysis image is segmented from the corrected reflected image to include a scene of interest for analysis. The method and apparatus disclosed in the '120 patent or the '939 patent employs Beer's law to determine such characteristics as the hemoglobin concentration per unit volume of blood, the number of white blood cells per unit volume of blood, a mean cell volume, the number of platelets per unit volume of blood, and the hematocrit.

To accurately determine the blood characteristics, however, the images need to be screened to identify images having good measurable properties. The measurements taken from the images also need to be screened, normalized and corrected to obtain better estimates of the true value of the blood characteristics.

Thus, there is a need in the art for a method and device that selects images having good measurable properties and provides reliable, quantitative estimates of blood cells, normal and abnormal contents of blood cells, and normal and abnormal constituents of blood plasma by using non-invasive in vivo analysis.

### SUMMARY OF THE INVENTION

The present invention is directed to screening reflected spectral images of a microcirculatory system to determine whether the spectral image is suitable for hemoglobin measurement and prediction. The method and apparatus of the present invention analyze the spectral image (also referred to herein as “blood sample” or “image”) to identify images having good measurable properties.

In an embodiment, multi-stage screening is performed on each input image to determine whether the image is suitable for hemoglobin measurement and prediction. During the screening process the following quantities are measured: image intensity, image background intensity variation, image focus condition, image motion blur condition, number of vessels in the image with certain range of diameters, average vessel edge contrast, number of bubble segments in the image and the similarity between two images.

The multi-stage screening cycle includes intensity screening, discrete wavelet transform (DWT) decomposition, focus measurement, motion blur measurement, vessel and bubble detection, and repetition detection. During the screening stages, the image is classified into one of three categories: excellent, mediocre and poor. Only the images having an excellent rating is used for further Hb estimation. But both excellent and mediocre images would launch an autofocusing process provided by focus measurement.

In another embodiment, a Fast Fourier transform is used to develop a spectral ratio to detect excellent images. The Fast Fourier transform is used to compute a power spectrum from the image. The spectral ratio is derived by computing a ratio from the slopes of the power spectrum at low and intermediate spatial frequencies. If the spectral ratio falls within a predetermined range, the image is judged as having good measurable properties.

The present invention provides real time hemoglobin prediction on the subject under investigation. The method is used to perform in vivo analysis of blood in large vessels, and in vivo analysis of blood in small vessels to determine blood parameters such as concentrations and blood cell counts. The method of the present invention can also be used to conduct non-invasive in vivo analysis of non-cellular characteristics of capillary plasma. The method of the present invention can also be used to perform in vitro analyses by imaging blood in, for example, a tube or flow cell.

The method of the present invention can also be used to analyze other types of fluids containing visualizable components. The reflected spectral imaging system can be used to analyze fluids for particulate impurities. It is only necessary that the walls of the fluid path be sufficiently transparent to permit light to pass through the walls of the fluid path to image the fluid and any impurities flowing in the path.

### BRIEF DESCRIPTION OF THE DRAWINGS

The present invention is described with reference to the accompanying drawings. En the drawings, like reference numbers indicate identical or functionally similar elements. Additionally, the left-most digit(s) of a reference number identifies the drawing in which the reference number first appears.

FIG. 1 shows a flow chart representing the general operational flow for screening spectral images according to an embodiment of the present invention;

FIG. 2 shows a flow chart illustrating step 104 shown in FIG. 1;

FIG. 3 shows a flow chart illustrating step 110 shown in FIG. 1;

FIG. 4 shows a flow chart illustrating step 112 shown in FIG. 1;

FIG. 5 shows a flow chart illustrating step 114 shown in FIG. 1;

FIG. 6 shows a flow chart illustrating step 116 shown in FIG. 1;

FIG. 7 shows a flow chart illustrating step 118 shown in FIG. 1;

FIG. 8 shows a flow chart illustrating step 120 shown in FIG. 1;

FIG. 9 shows a flow chart illustrating step 122 shown in FIG. 1;

FIG. 10 shows a flow chart illustrating step 124 shown in FIG. 1;

FIG. 11 shows a flow chart illustrating steps 1026 and steps 1028 shown in FIG. 10;

FIG. 12 shows a flow chart illustrating step 1128 shown in FIG. 11;

FIG. 13 shows a flow chart illustrating step 126 shown in FIG. 1;

FIG. 14 shows a flow chart representing the general operational flow for screening spectral images according to another embodiment of the present invention; and

FIG. 15 is a block diagram of an example computer system useful for implementing the present invention.

### DETAILED DESCRIPTION OF THE PREFERRED EMBODIMENTS

The present invention is directed to a method and apparatus for analysis, particularly non-invasive, in vivo analysis of a subject's vascular system. The in vivo measurements discussed herein can also be performed in vitro by imaging blood in, for example, a tube or flow cell, as would be apparent to a person skilled in the relevant art(s). The in vivo method is carried out by imaging a portion of the subject's vascular system. For example, the image can be created from a sub-surface region of a subject's tissues or organs. The tissue covering the imaged portion must be traversed by light without multiple scattering to obtain a reflected image. In order to form an image, two criteria must be met. First, there must be image contrast resulting from a difference in the optical properties, such as absorption, index of refraction, or scattering characteristics, between the subject to be imaged and its surroundings or background. Second, the light that is collected from the subject must reach an image capturing means without substantial scattering, i.e., the reflected image must be captured from a depth that is less than the multiple scattering length. As used herein, “image” refers to any image that satisfies the foregoing two criteria. As used herein, “reflected image” refers to the image of a subject in reflected light. The resolution required for capturing the image is dictated by the spatial homogeneity of the imaged portion. For example, a reflected image of individual cells requires high resolution. A reflected image of large vessels can be done with low resolution. A reflected image suitable for making a determination based on pallor requires very low resolution.

The tissue covering the imaged portion is thus preferably transparent to light, and relatively thin, such as the mucosal membrane on the inside of the lip of a human subject. As used herein, “light” refers generally to electromagnetic radiation of any wavelength, including the infrared, visible, and ultraviolet portions of the spectrum. A particularly preferred portion of the spectrum is that portion where there is relative transparency of tissue, such as in the visible and near-infrared wavelengths. It is to be understood that for the present invention, light can be coherent light or incoherent light, and illumination may be steady or in pulses of light.

The reflected image is corrected to form a corrected reflected image. The correction to the reflected image is done, for example, to isolate particular wavelengths of interest, or to extract a moving portion of the image from a stationary portion of the image. A scene is segmented from the corrected reflected image to form an analysis image. The analysis image is then analyzed for the desired characteristic of the subject's vascular system.

The method of the present invention can be used for analysis in large and small vessels, including capillary plasma. As used herein, “large vessel” refers to a vessel in the vascular system of sufficient size so that a plurality of red blood cells flow side-by-side through it. “Small vessel” refers to a vessel in the vascular system of a size so that red blood cells flow substantially “single file” through it. As explained in more detail below, the present invention uses reflectance, not transmission, for the images that are analyzed. That is, the image is made by “looking at” the vascular system, rather than by “looking through” the vascular system. However, because of the features of the imaging system used in the present invention, as described in detail in the above-referenced '120 patent or the '939 patent, the image appears to be of the transmission type. For this reason, Beer's law can be applied to quantitatively measure the images. Per unit volume or concentration measurements can be made directly from the images. Therefore, although the present invention uses reflectance, it would be apparent to a person skilled in the relevant art(s) that the method of the present invention can be used on both transmitted and reflected images.

By using the method of the present invention to provide a reflected spectral image of large vessels, the hemoglobin (Hb), hematocrit (Hct), and white blood cell count (WBC) parameters can be directly determined. By using the method of the present invention to provide a reflected spectral image of small vessels, mean cell volume (MCV), mean cell hemoglobin concentration (MCHC), and platelet count (Plt) can be directly determined.

Human blood is made up of formed elements and plasma. There are three basic types of formed blood cell components: red blood cells (erythrocytes); white blood cells (leukocytes); and platelets. As noted above, red blood cells contain hemoglobin that carries oxygen from the lungs to the tissues of the body. White blood cells are of approximately the same size as red blood cells, but do not contain hemoglobin. A normal healthy individual will have approximately 5,000,000 red blood cells per cubic millimeter of blood, and approximately 7,500 white blood cells per cubic millimeter of blood. Therefore, a normal healthy individual will have approximately one white blood cell for every 670 red blood cells circulating in the vascular system.

A complete blood count (CBC) without white blood cell differential measures eight parameters: (1) hemoglobin (Hb); (2) hematocrit (Hct); (3) red blood cell count (RBC); (4) mean cell volume (MCV); (5) mean cell hemoglobin (MCH); (6) mean cell hemoglobin concentration (MCHC); (7) white blood cell count (WBC); and (8) platelet count (Plt). The first six parameters are referred to herein as RBC parameters. Concentration measurements (measurements per unit volume of blood) are necessary for producing values for Hb, Hct, RBC, WBC, and Plt. Hb is the hemoglobin concentration per unit volume of blood. Hct is the volume of cells per unit volume of blood. Hct can be expressed as a percentage, i.e.,:

(cell volume÷volume of blood)×100% (Eqn. 1)

RBC is the number of red blood cells per unit volume of blood. WBC is the number of white blood cells per unit volume of blood. Plt is the number of platelets per unit volume of blood.

Red cell indices (MCV, MCH, and MCHC) are cellular parameters that depict the volume, hemoglobin content, and hemoglobin concentration, respectively, of the average red cell. The red cell indices may be determined by making measurements on individual cells, and averaging the individual cell measurements. Red cells do not change volume or lose hemoglobin as they move through the vascular system. Therefore, red cell indices are constant throughout the circulation, and can be reliably measured in small vessels. The three red cell indices are related by the equation:

MCHC=MCH÷MCV (Eqn. 2)

Thus, only two red cell indices are independent variables.

To determine values for the six RBC parameters listed above, the following two criteria must be met. First, three of the parameters must be independently measured or determined. That is, three of the parameters must be measured or determined without reference to any of the other of the six parameters. Second, at least one of the three independently measured or determined parameters must be a concentration parameter (per unit volume of blood). Therefore, values for the six key parameters can be determined by making three independent measurements, at least one of which is a concentration measurement which cannot be made in a small vessel.

As disclosed in the '120 patent or the '939 patent, Hb and Hct can be directly measured by reflected spectral imaging of large vessels, and MCV can be directly measured by reflected spectral imaging of small vessels. In this manner, three parameters are independently measured, and two of the parameters (Hb and Hct) are concentration parameters measured per unit volume of blood. As such, the six RBC parameters listed above can be determined in the following manner:

| | |
|---|---|
| Hb | Directly measuredc |
| Hct | Directly measured |
| RBC | Hct ∓ MCV |
| MCV | Directly measured |
| MCH | MCV × (Hb ∓ Hct) |
| MCHC | Hb ∓ Hct |

Also, as disclosed in the '120 patent or the '939 patent, Hb can be directly measured by reflected spectral imaging of large vessels, and MCV and MCHC can be directly measured by reflected spectral imaging of small vessels. In this manner, three parameters are independently measured, and one of the parameters (Hb) is a concentration parameter measured per unit volume of blood. As such, the six RBC parameters listed above can be determined in the following manner:

| | |
|---|---|
| Hb | Directly measured |
| Hct | Hb ∓ MCHC |
| RBC | Hb ∓ (MCV × MCHC) |
| MCV | Directly measured |
| MCH | MCV × MCHC |
| MCHC | Directly measured |

Concentration measurements are measurements per unit volume. As discussed above, a measurement made per unit area is proportional to a measurement made per unit volume (volume measurement with constant depth) when the depth of penetration is constant. The depth of penetration is a function of wavelength, the size of the particles with which it interacts, and refractive index. For blood, the particle size and index of refraction are essentially constant. Consequently, the depth of penetration will be constant for a particular wavelength.

Hemoglobin is the main component of red blood cells. Hemoglobin is a protein that serves as a vehicle for the transportation of oxygen and carbon dioxide throughout the vascular system. Hemoglobin absorbs light at particular absorbing wavelengths, such as 550 nm, and does not absorb light at other non-absorbing wavelengths, such as 650 nm. Under Beer's law, the negative logarithm of the measured transmitted light intensity is linearly related to concentration. As explained more fully in the '120 patent or the '939 patent, a spectral imaging apparatus can be configured so that reflected light intensity follows Beer's law. Assuming Beer's law applies, the concentration of hemoglobin in a particular sample of blood is linearly related to the negative logarithm of light reflected by the hemoglobin. The more 550 nm light absorbed by a blood sample, the lower the reflected light intensity at 550 nm, and the higher the concentration of hemoglobin in that blood sample. The concentration of hemoglobin can be computed by taking the negative logarithm of the measured reflected light intensity at an absorbing wavelength such as 550 nm. Therefore, if the reflected light intensity from a particular sample of blood is measured, the concentration in the blood of such components as hemoglobin can be directly determined.

The method of the present invention can also be used to determine the hematocrit (Hct). The difference between hemoglobin (which is the grams of hemoglobin per volume of blood) and hematocrit (which is the volume of blood cells per volume of blood) is determined by the concentration of hemoglobin within the cells which determines the index of refraction of the cells. Hence, measurements in which the image contrast between the circulation and the background is achieved principally by the scattering properties of the circulation will be related to the hematocrit and those obtained principally by the absorbing properties will be related primarily to the hemoglobin. For example, the microvascular system beneath the mucosal membrane on the inside of the lip of a human subject can be imaged to produce a raw reflected image whose contrast is determined by a difference in the scattering properties of the blood cells.

As disclosed in the '120 patent or the '939 patent, a spectral imaging apparatus includes a light source that is used to illuminate the portion of the subject's vascular system to be imaged. The reflected light is captured by an image capturing means. Suitable image capturing means include, but are not limited to, a camera, a film medium, a photocell, a photodiode, or a charge coupled device camera. An image correcting and analyzing means, such as a computer, is coupled to the image capturing means for carrying out image correction, image screening, scene segmentation, and blood characteristic analysis.

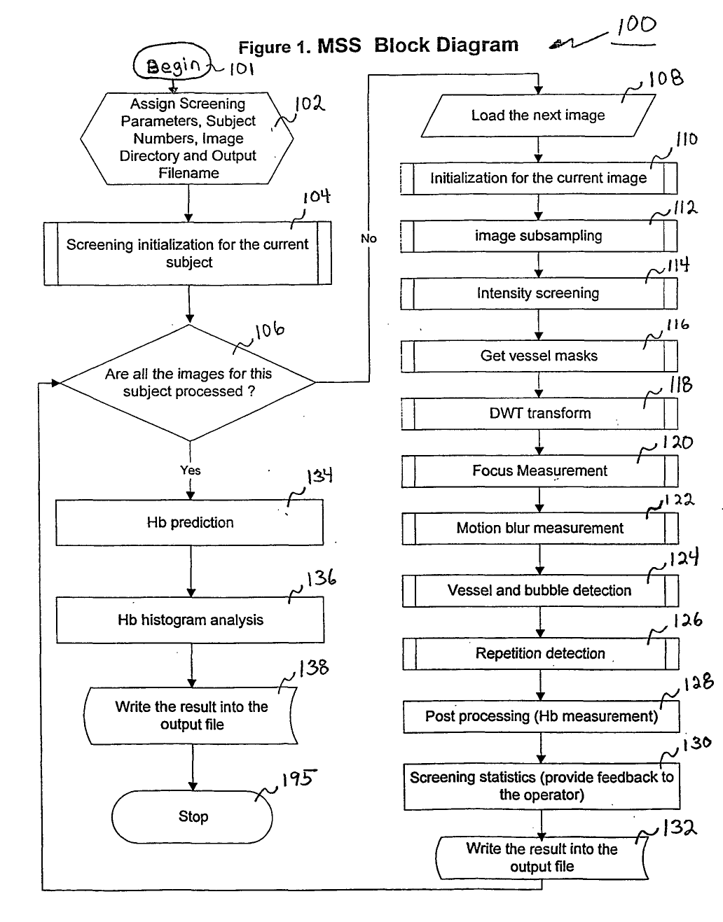

To implement the method of the present invention, a spectral image is captured and processed to select images having good measurable properties. The screened image(s) is analyzed to produce a reliable measurement of the concentration and volume of blood characteristics, such as hematocrit or hemoglobin. FIG. 1 illustrates a general operational flow of an embodiment of the present invention. More specifically, flowchart 100 shows an example of a process for screening a spectral image of a blood or tissue sample. Still more particularly, flowchart 100 describes a process for collecting information about the spectral image to better judge the quality of the image and perform vessel measurements to determine its characteristics. Although FIG. 1 is described in referenced to predicting hemoglobin, other blood characteristics can also be measured from the images selected by the present method.

The spectral image can be obtained from a spectral imaging apparatus preferably, but not necessarily, of the type described in the '120 patent or the '939 patent. Nonetheless, the spectral image can be obtained from any type of imaging apparatus designed for tissue or blood analysis, as would be apparent to a person skilled in the relevant art(s).

FIG. 1 starts at step 101 and passes immediately to step 102, where spectral images are retrieved from a memory source or image directory. The images can be retrieved from an input file stored in a temporary or permanent memory location on a hard disk drive or removable storage device, such as a floppy diskette, magnetic tape, optical disks, or the like. The input file also includes the subject number or other data used to identify the subject or patient. At step 102, an output file is also created to store relevant test results, as described below in further detail.

Also, at step 102, screening parameters are downloaded for use during the subsequent screening steps. The screening parameters include the width and height for an original image. In an embodiment, 8-bit images are used for the screening process according to the present invention. Using 8-bit images maximizes performance requirements while maintaining accuracy. Higher dynamic range images can be used but may degrade the processing speed.

Other screening parameters include the preset size for cropped images and position within the original image; Discrete Wavelet Transform (DWT) decomposition level; mask size for use during repetition detection (as described below); search range for use during repetition detection; number of sampled rows and columns for screening and the sampling positions (as described below); and various threshold values. The threshold values include SCREEN_IM_MIN, SCREEN_IM_MAX, SCREEN_IV_MAX, SCREEN_MB_THRESH, SCREEN_VE_MIN_D, SCREEN_VE_MAX_D, SCREEN_VN_THRESH, SCREEN_BN_THRESH,SCREEN RP_THRESH and SCREEN_FO_THRESH.

The threshold values SCREEN_IM_MIN and SCREEN_IM_MAX set the minimum and maximum intensity levels, respectively. If an image's mean intensity exceeds this range, the image would not pass the screening steps as discussed below. Another threshold value is SCREEN_IV_MAX, which determines the maximum allowed background intensity variation value. If an image has a background intensity variation larger than this value, the image would not pass screening. The SCREEN_MB_THRESH threshold value sets the motion blur threshold. Images with motion blur measurement larger than this value would not pass screening.

The threshold values SCREEN_VE_MIN_D and SCREEN_VE_MAX_D represent the minimum and maximum vessel diameters, respectively. If a vessel has a diameter beyond this range, the vessel measurements would not be counted. The threshold value SCREEN_VN_THRESH is the number of vessel segment with an appropriate size, meaning that images having a number of vessel segments less than this value would not pass the screening. The threshold value SCREEN_BN_THRESH sets the number of bubble segments. The images having a number of bubble segments larger than this value would not pass screening.

SCREEN_RP_THRESH is the repetition threshold. Two images having a repetition parameter larger than this value would be considered from the same site. SCREEN_FO_THRESH is the minimum vessel edge contrast threshold. The edge contrast threshold is set adaptively. However during the initial screening cycle, this value is set as the threshold because historical values would not be in existence.

At step 104, the screening process is initialized for the subject and the subject's images are selected. At step 106, the first image, if any, is selected and at step 108, the image is loaded for screening. At step 110, the screening process is initialized for the loaded image. At step 112, the image is subsampled for real time screening. Subsampling increases the processing speed by uniformly extracting a predetermined number of rows and columns from the image. In an embodiment, sixteen rows and columns are subsampled from the image. However, the present invention can be practiced with any number up to the total number of pixel rows and columns in the image. Al screening information is obtained from this data.

Step 114 is the first screening step. At step 114, the subsampled images are evaluated to determine the mean image intensity value and intensity variation value. Statistical information (such as, minimum, maximum and mean values) is computed for the rows and columns within each subsampled image. The mean intensity of all the samples approximates the mean intensity of the whole image. The standard deviation among the mean intensities of the sampled rows and columns is used to approximate the intensity variation of the image. These calculations are used to determine whether the intensity of the image under inspection is within a linearity range of the illumination and whether no intensity background shadowing is present. If the images are saturated, Beer's law may not hold. Thus, too bright or too dark images are not considered as good images. These calculations are also used to ensure that no big background intensity variation is present in the selected images. In most cases, the shadow from deeper vessels would cause significant intensity variation which would influence the hemoglobin prediction. Thus, images with large background intensity variation should not be selected.

At step 116, a binary vessel mask is generated for the current image. This mask would be used for repetition detection (discussed in detail below) to determine whether the current image is from the same site as a controlled image. The controlled image, typically, is the previously processed image. For the initial screening cycle, the controlled image can be determined from an initialization parameter entered at step 102. To increase the processing speed, two image masks (row mask and column mask) are generated. When generating these masks, the cropped image is divided into 3×3 blocks. Each block would have its own threshold for background and vessel segmentation. Using two image masks reduces the influence of bowl-shape background illumination pattern.

At step 118, discrete wavelet transform (DWT) is performed on the image so as to use scale information as well as spatial information for the screening. Based on the DWT decomposition level, DWT decomposes the image to derive wavelet coefficients for the input rows and columns.

At step 120, focus measurement is computed from the high frequency components of the images in an appropriate scale. The focus measurement is used to compare the focus conditions of two images from the same scene. The focus measurement is used to exclude those images which are out of focus. This automatic focusing routine is implemented by analyzing the average vessel edge contrast of each image. If the average edge contrast of an image is much lower than the controlled image (or, alternatively, the running average of previous selected images), the current image would be discarded even if it had passed other screening criteria.

At step 122, a motion blur measurement is computed for the current image. The measurement is compared against the motion blur threshold entered at step 102. Images having motion blur measurement that exceeds this threshold would not be used for Hb measurements. Motion blur usually reduces the contrast of the vessels and also causes inaccurate vessel diameters measurement.

At step 124, vessel and bubble detection is performed. If bubbles are detected in the image or if no good sized vessels can be located, the image would not be used for further processing. The bubbles (typically, caused by liquids, such as water or saliva) would usually produce intensity distortion on the vessels. As for vessel detection, the diameter of each vessel segment is measured for each detected vessel. Only the vessels whose diameter is within a specified range would be counted as good-sized vessels.

At step 126, the focus measurement and vessel masks are analyzed for repetition detection to determine if the current image and controlled image are from the same site. Repetition detection ensure that only one image is kept for each distinct site. Therefore, repetition detection ensures that Hb measurements are based on each site, not each image. It is presumed that each distinct site has the same contribution towards the final hemoglobin prediction. If the currently selected image has been detected to be similar enough with the controlled image (typically, the previously selected image), only the one with higher focus measurement would be saved for further measurement. Step 126 is the final screening step.

At step 128, an overall screening summary is prepared and/or updated for the current subject. The summary also includes a running average of all information for the current subject, and site information for the current subject. At this step, adaptive thresholds are also generated for further screening. Moreover, distinct sites are determined from the screening information. For instance, a distinct site would exist if there was a big motion, a sequence of bad images (i.e., images not passing the screening steps 114-126) or the size of the current site exceeds a certain threshold.

At step 130, the screening information or statistics are reported to an operator. The information can be displayed on a display unit, presented in a paper report, or the like. The screening information allows the operator to determine if the spectral imaging apparatus, or its light source or probe, is being moved in an appropriate way. In an embodiment, the screening results of the last fifty images are evaluated. If too much repetition is determined at step 126, this is an indication that the probe is moving too slow. If too much motion blur is detected at step 122, the probe is moving too fast. If too many images fail bubble screening at step 124, there are too many bubbles in the scene, which must be eliminated.

At step 132, the screening information is stored in the output file, and at step 106 the next image is selected to repeat the screening and processing steps 108-132. If, however, no more images are available for processing, the control flow passes to step 134.

At step 134, the images passing the screening thresholds are selected for each site. In an embodiment, only one image is selected from each site for further processing. The selected images should be distortion free and contain good-sized vessels for hemoglobin prediction. To predict the hemoglobin, the value OD/D is computed for each vessel segment in the images. D represents the vessel diameter. OD is the optical density and can be computed by OD=log10(Ib÷Iv), where Iv is the mean vessel intensity and Ib is the mean background intensity for each vessel segment. Other vessel segment information includes direction, contrast and position. In an embodiment, OD/D is computed for each image at step 128. In another embodiment, OD/D is computed only for the selected images at step 134.

At step 136, a histogram analysis of the Hb predictions are performed. On the histogram, frequency is plotted as a function of Hb. The histogram is used to select the best and most reasonable image. At step 138, the results are stored in the output file. The control flow, then, ends as indicated by step 195.

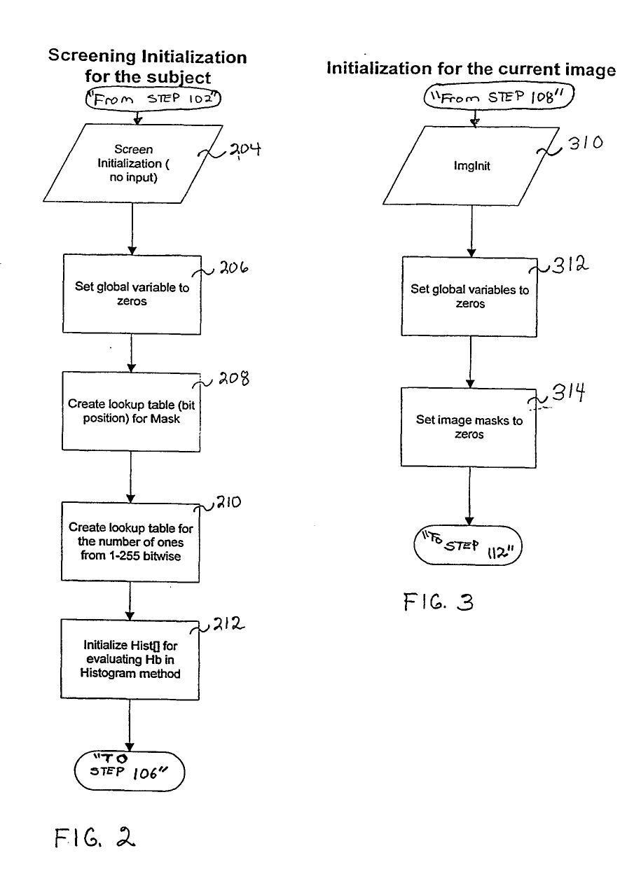

FIG. 2 illustrates an operational flow of an embodiment for screening initialization for a current subject. More specifically, FIG. 2 is an embodiment of a more detailed description of process step 104 from FIG. 1. At step 204, the screening initialization process is started by allocating memory space for processing. At step 206, all global variables are set or reset to zero, depending on whether it is the initial or subsequent screening cycle. The global variables are the screening parameters determined at screening steps 114-126 and are used to evaluate images for selection. At step 208, a lookup table is generated to increase the ability to quickly process the masks generated during the screening steps. At step 210, a lookup table is generated for the number of ones from 1 to 255 bitwise. At step 212, a histogram file is opened for plotting Hb measurements at step 136. Then the control flow passes to step 106, as previously described.

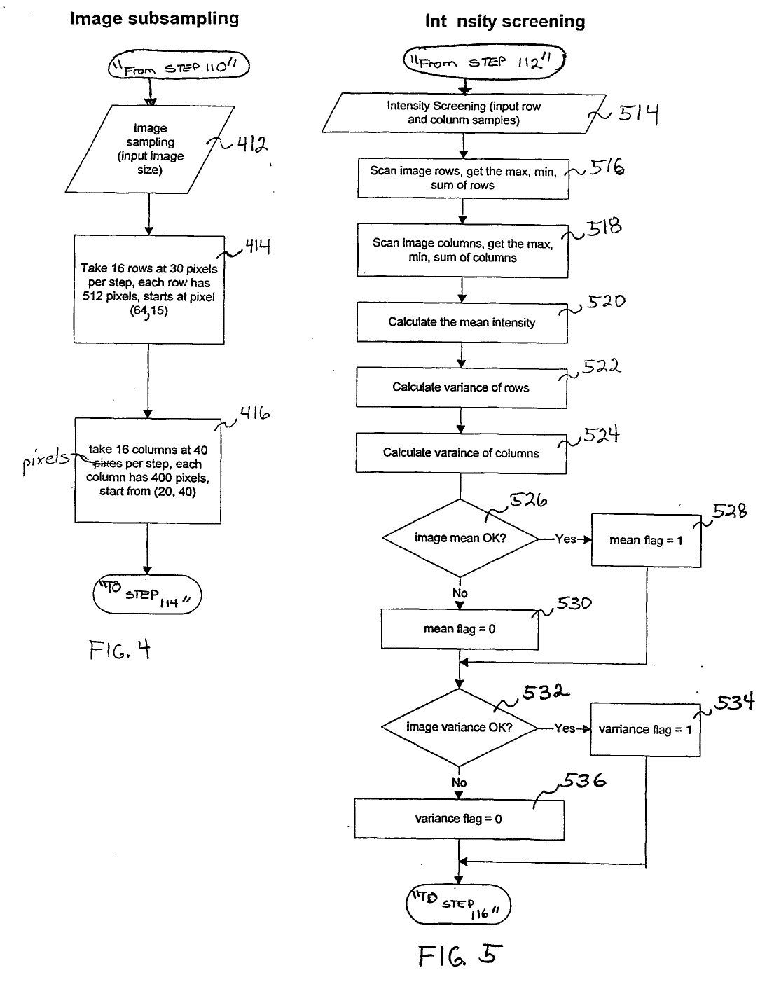

FIG. 3 illustrates an operational flow of an embodiment for screening initialization for a currently loaded image. More specifically, FIG. 3 is an embodiment of a more detailed description of process step 110 from FIG. 1. At step 310, buffers are setup to analyze the current image. At step 312, the global variables are set or reset to zero, depending on whether the current image is the first image to be processed for the current subject. At step 314, the image masks are also set or reset to zero. Then the control flow passes to step 112, as previously described.

FIG. 4 illustrates an operational flow of an embodiment for subsampling a current image. More specifically, FIG. 4 is an embodiment of a more detailed description of process step 112 from FIG. 1. At step 412, the current image and the input parameters are received. The input parameters include the subsample step and position, and the number of subsampled horizontal and vertical lines. These parameters are entered at step 102. At step 414, sixteen rows are uniformly extracted from the image. In an embodiment, the extraction occurs at thirty pixels per step, with each row having 512 pixels. In an embodiment, the extraction starts at pixel (64, 15).

At step 416, sixteen columns are uniformly extracted from the image. In an embodiment, the extraction occurs at forty pixels per step, with each column having 400 pixels. In an embodiment, the extraction starts at pixel (20, 40). The subsampled horizontal and vertical lines are outputted as global variables. Then the control flow passes to step 114, as previously described.

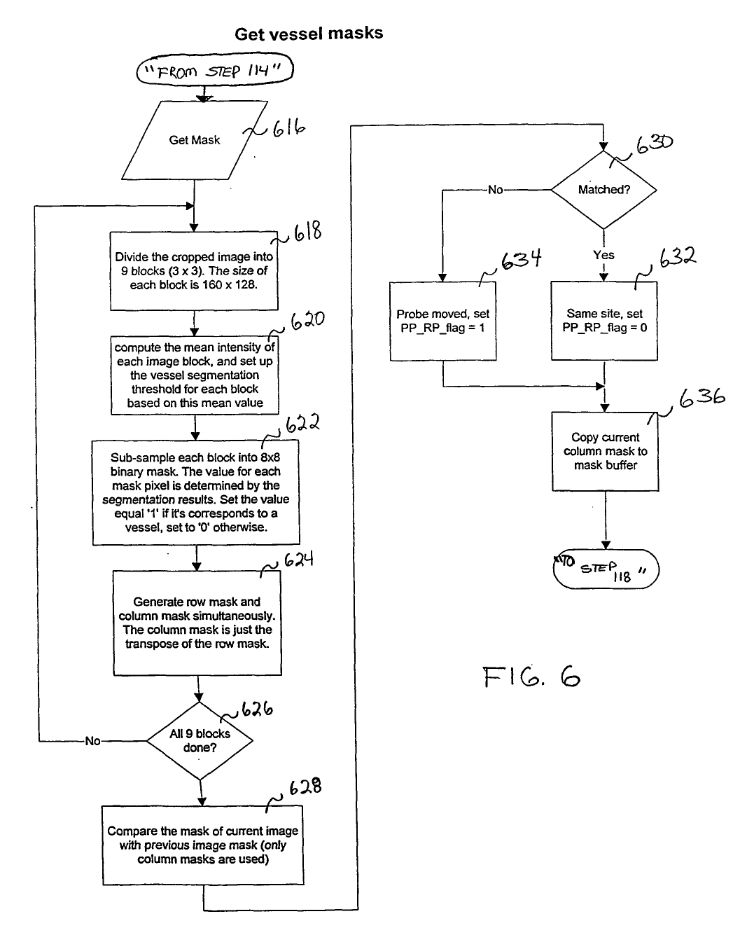

FIG. 5 illustrates an operational flow of an embodiment for intensity screening a current image. More specifically, FIG. 5 is an embodiment of a more detailed description of process step 114 from FIG. 1. At step 514, the subsampled rows and columns from step 112 are received as global variables for analysis. At step 516, each subsampled row is scanned to compute a maximum, minimum and sum of the intensity. At step 518, each subsampled column is scanned to compute a maximum, minimum and sum of the intensity. At step 520, a mean intensity for the whole image is calculated from all subsampled mean intensities. At step 522, a row variance and/or standard deviation for the whole image is calculated from the mean intensities of the subsampled rows. At step 524, a column variance and/or standard deviation for the whole image is calculated from the mean intensities of the subsampled columns.

At steps 526 and 532, mean intensity, row variance (or standard deviation) and column variance (or standard deviation) are compared to the minimum and maximum intensity threshold values (SCREEN_IM_MIN and SCREEN_IM_MAX) entered at step 102. If the calculated values fall within the range set by the threshold values, a mean or variance flag is set to “1” at step 528 or step 534, as appropriate. If the calculated values fall outside of the range, the mean or variance flag is set to “0” at step 530 or step 536, as appropriate. The mean image intensity value, intensity variation value, and intensity screening status (i.e., mean or variance) flag(s) are outputted, and the control flow passes to step 116, as previously described.

FIG. 6 illustrates an operational flow of an embodiment for generating a binary vessel mask for a current image. More specifically, FIG. 6 is an embodiment of a more detailed description of process step 116 from FIG. 1. At step 616, the current image and the screening parameter for the size of cropped image and its position (from step 102) are received. The mask of a controlled image is also received. The controlled image can be the previously screened image, or it can be a screening parameter inputted at step 102. At step 618, the cropped image is divided into nine blocks (i.e., three-by-three). The size of each block is 160 by 128.

At step 620, the mean intensity is computed for each image block. A vessel segmentation threshold is established for each block based on the mean intensity. At step 622, each block is subsampled into an eight-by-eight binary mask. The value for each mask pixel is determined by the segmentation results. The value would be set to “1” if the pixel corresponds to a vessel and “0” if otherwise. At step 624, a row mask and column mask is generated simultaneously. The column mask is determined as the transpose of the row mask. At step 626, the process is repeated for all nine image blocks.

At step 628, the mask for the current image is compare to the mask for the controlled image. In an embodiment, only the column masks are used. At step 630, it is determined whether the compared masks match each other. If a match is determined, at step 632, a site flag is set to “0” to indicate the images are from the same site. If no match is determined, at step 634, a site flag is set to “1” to indicate the images are from a different site. Accordingly, the probe can be relocated to take images from another site.

At step 636, the mask for the current image is copied to a mask buffer. In an embodiment, this mask would be used as the controlled mask for screening subsequent images. Then the control flow passes to step 118, as previously described.

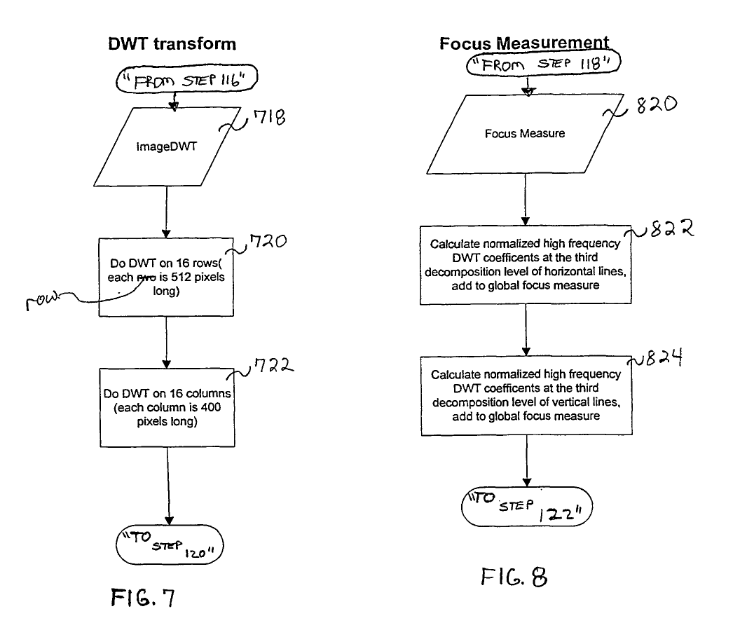

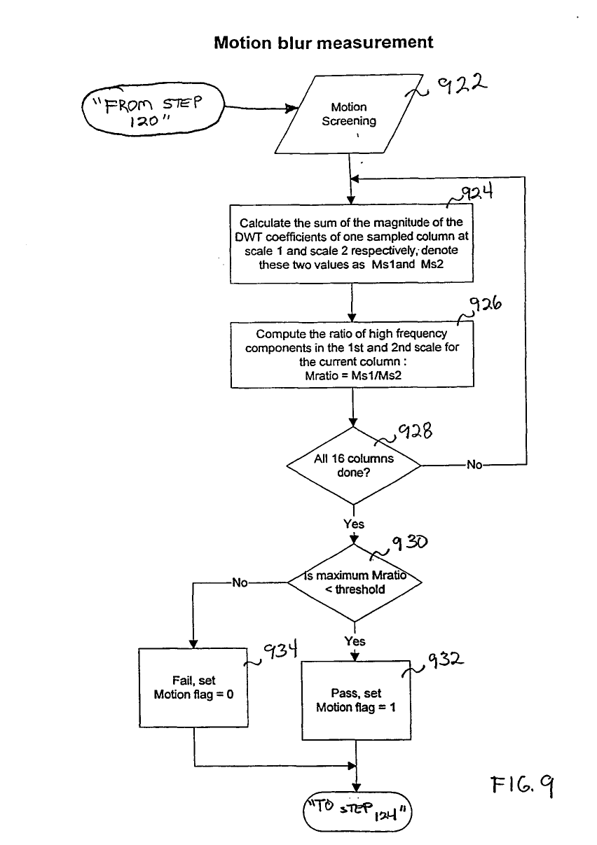

FIG. 7 illustrates an operational flow of an embodiment for performing a DWT transform on a current image. More specifically, FIG. 7 is an embodiment of a more detailed description of process step 118 from FIG. 1. At step 718, the following global variables are received for processing: the subsampled rows and columns and their lengths (from step 112), and the DWT decomposition level (from step 102). At step 720, a one-dimensional circular DWT is computed on each of the sixteen subsampled rows, with each row being 512 pixels in length. At step 722, a one-dimensional circular DWT is computed on each of the sixteen subsampled columns, with each column being 400 pixels in length. The output of the DWT computations is a coefficient vector with the same length as the inputted data. The DWT coefficients have a multi-scale structure. In a preferred embodiment, a decomposition level of three is used, with a scale of“1” referring to the finest scale, “2” referring to the medium scale, and “3” referring to the coarsest scale. Following the DWT computations, the control flow passes to step 120, as previously described.

FIG. 8 illustrates an operational flow of an embodiment for determining the focus measurement from a current image. More specifically, FIG. 8 is an embodiment of a more detailed description of process step 120 from FIG. 1. At step 820, the following global variables are received: the DWT coefficients for the subsampled rows and columns and their lengths (from step 118), mean intensity of each sampled row and column (from step 114), and DWT decomposition level (from step 102). In an embodiment, the high frequency coefficients at the third decomposition level is used as the focus measurement. The measurement is normalized by using the average mean intensity for the current image. The DWT coefficients at the third level reflect the edge sharpness of larger vessels, at the same time, they are less sensitive to the noise than the coefficients at finer scales, i.e. “1” and “2.”

Accordingly, at step 822, the high frequency DWT coefficients at the third decomposition level of horizontal lines are normalized and added to the focus measurement. At step 824, the high frequency DWT coefficients at the third decomposition level of vertical lines are normalized and added to the focus measurement. Then the control flow passes to step 122, as previously described.

FIG. 9 illustrates an operational flow of an embodiment for measuring motion blur for a current image. More specifically, FIG. 9 is an embodiment of a more detailed description of process step 122 from FIG. 1. At step 922, the global parameters are received, namely the DWT coefficients for the subsampled rows and columns and their lengths (from step 118), and the DWT decomposition level (from step 102). At step 924, a calculation is made of the sum of the magnitude of the DWT coefficients of a subsampled column at scale 1 and scale 2, respectively. The calculated values are denoted as Ms1 and Ms2.

At step 926, a ratio of Ms1 to Ms2 is computed for high frequency components in the first and second scale for the current column. At step 928, the process defined in steps 924-926 is repeated for all sixteen subsampled columns. At step 930, the maximum ratio is determined and compared to the motion blur threshold value from step 102. If the maximum ratio is less than the threshold value, at step 932, a motion flag is set to “1” to designate the image has passed. Otherwise, at step 934, the motion flag is set to “0” to designate the image has failed the motion blur screening.

This embodiment for motion blur screening is premised on the assumption that motion generally produces a shift between two fields in an image. The shift would generate a large amount of high frequency components for the subsampled columns, and the high frequency components would disappear in the coarser scale because of the subsampling. Therefore, the ratio of Ms1 to Ms2 is a good indicator of motion blur. The motion blur measurements and motion blur screening status flag are outputted as global variables, and the control flow passes to step 124, as previously described.

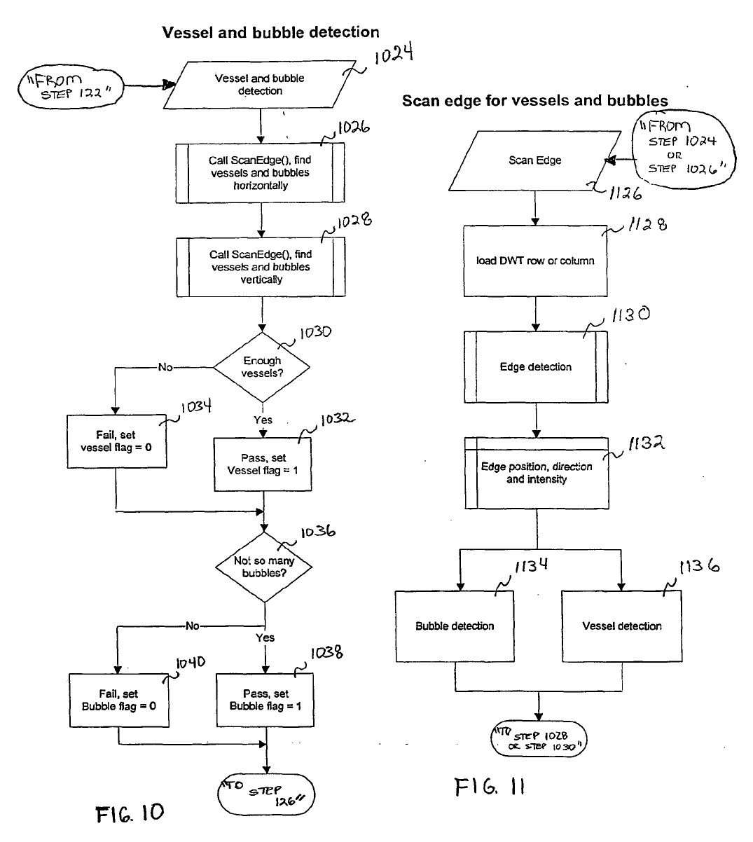

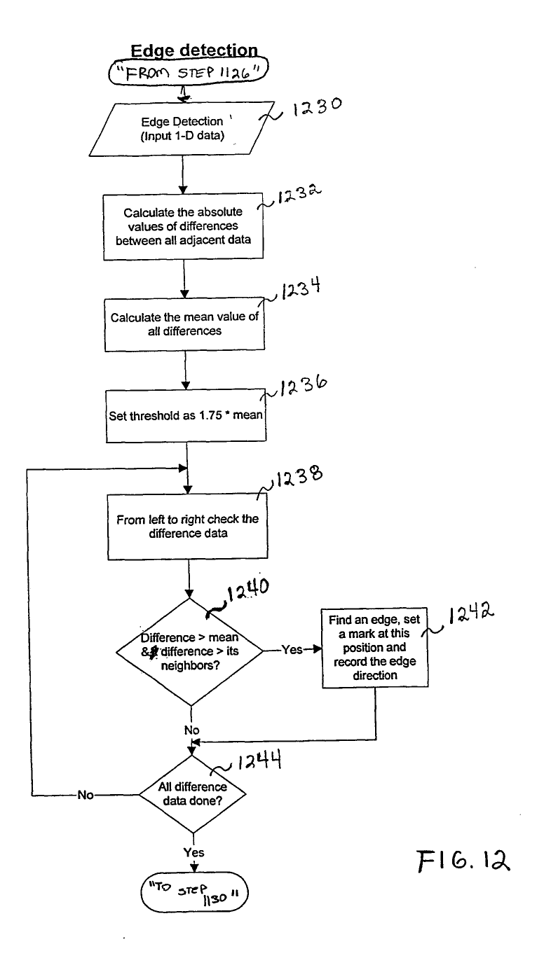

FIG. 10 illustrates an operational flow of an embodiment for performing vessel and bubble detection on a current image. More specifically, FIG. 10 is an embodiment of a more detailed description of process step 124 from FIG. 1. At step 1024, the global parameters are received for processing. The parameters include the DWT coefficients of subsampled rows and columns, the DWT decomposition level, the original input images and the sizes of the input data. At step 1026, horizontal edge scanning is performed to detect vessel and bubble segments in the image. At step 1028, vertical edge scanning is performed to detect vessel and bubble segments in the image. At step 1030, the detected vessels are analyzed to determine if the vessel size is sufficient. At step 1032, a vessel flag is set to “1” if a predetermined number of good-sized vessels are detected. Otherwise, at step 1034, the vessel flag is set to “0.”

At step 1036, the bubble segments, if any, are evaluated. At step 1038, a bubble flag is set to “1” if the number of detected bubbles exceed a preset threshold. Otherwise, at step 1040, the bubble flag is set to “0.” The output data for the subsequent steps include the number of vessel segments, number of bubble segments, vessel segment information, average edge contrast and the vessel and bubble screening status flags. Afterwards, the control flow passes to step 126, as previously described.

FIG. 11 illustrates an operational flow of an embodiment for the edge scanning process for a current subject. More specifically, FIG. 11 is an embodiment of a more detailed description of step 1026 and step 1028 from FIG. 10. The appropriate global parameters are obtained at step 1126. At step 1128, subsampled rows (for step 1026) or columns (for step 1028) are loaded for processing. At step 1130, edge detection is performed on each individual subsampled row or column, as appropriate, to obtain edge information, including edge position, direction and intensity or contrast. At step 1132, the edge information is recorded. At step 1134, the edge information is used to detect bubble segments in the image. At step 1136, the edge information is used to detect vessel segments in the image. Then the control flow passes to step 1028 or step 1030, as appropriate, as previously described.

FIG. 12 illustrates an operational flow of an embodiment for performing edge detection on a current image. More specifically, FIG. 12 is an embodiment of a more detailed description of process step 1130 from FIG. 11. At step 1230, the data is received from the loaded DWT rows or columns (from step 1128). Edge detection is based on gradient information and uses the low frequency band of the DWT coefficients. Accordingly, at step 1232, the absolute values are calculated for the differences between all adjacent data in the loaded DWT rows or columns. At step 1234, the mean value of all differences are calculated. At step 1236, a threshold value is derived from the mean value. In an embodiment, the threshold value is calculated as the product of 1.75 and the mean value.

At step 1238, the difference data is checked from left to right to determine the edge location, direction and edge strength. At step 1240, the difference data is compared to its neighbor. If the difference data exceeds the mean value and its neighbor's mean value, the control flow passes to step 1242. At step 1242, the edge location is determined, a mark is set at this position, and the edge direction is recorded. The control flow then passes to step 1244.

If, on the other hand, the difference data does not exceed the mean value and it neighbor's mean value, the control flow passes to step 1244. At step 1244, it is determined if all difference data has been processed. If not, the cycle repeats at step 1238. Otherwise, the control flow passes to step 1132, as previously described.

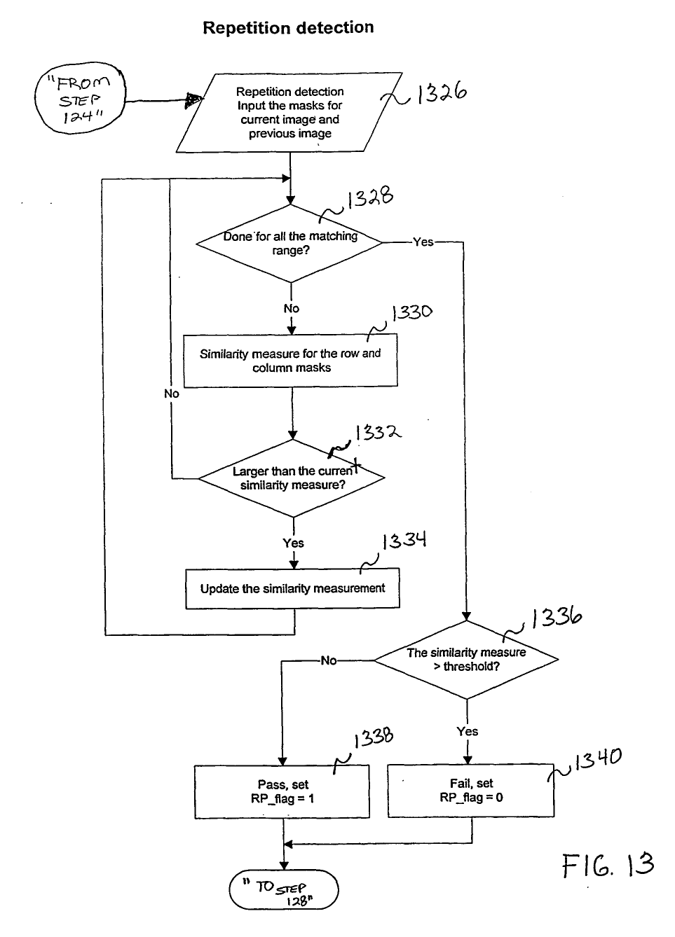

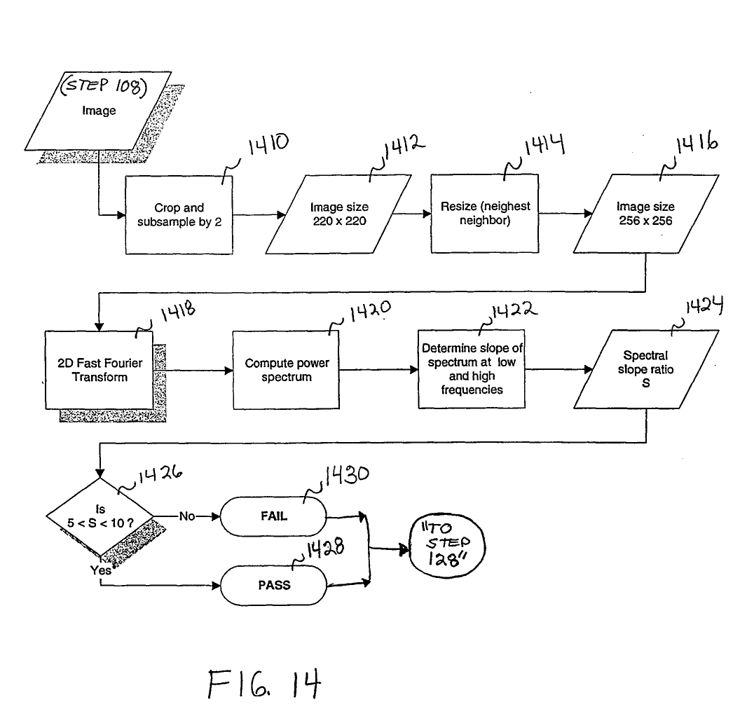

FIG. 13 illustrates an operational flow of an embodiment for measuring repetition detection for a current image. More specifically, FIG. 13 is an embodiment of a more detailed description of process step 126 from FIG. 1. At step 1326, the input parameters are received. The parameters include the vessel masks (row and column masks) of the current and controlled image, the matching range which is the searching range for maximum correlation between the two masks, the focus measurements of the two images, and the repetition threshold.

At step 1328, the matching range is evaluated to determine if repetition detection has been implemented over the specified range. At step 1330, the row and column masks for the current and controlled images are analyzed to detect similarities. At step 1332, the current similarity measurement is compared to the previous similarity measurement, and the larger value is recorded at step 1334. For the initial pass or cycle, the current similarity value would be recorded.

After the entire matching range had been evaluated, at step 1336, the similarity measurement is compared to the repetition threshold. If the repetition threshold is exceeded, at step 1340, a repetition flag is set to “0” to designate the image as failed because it is located at the same site as the controlled image. Otherwise, at step 1338, the repetition flag is set to “1” to designate the image represents a new and distinct site. The maximum similarity measurement and repetition screening flag is outputted, and the control flow passes to step 128, as previously described.

The above embodiments describe a multi-stage screening methodology for selecting images for spectral analysis. An alternative, single stage screening method is illustrated in FIG. 14. FIG. 14 is a general operational flow of another embodiment for screening spectral images. This embodiment utilizes a Fourier spectrum to select a subset of images that contain vascular structure. The control flow starts at step 108, where an image is loaded as described in reference to FIG. 1. At step 1410, the current image is cropped and subsampled according to a predetermined parameter, and the image size set at step 1412 as 220 by 220 pixels. At step 1414, the current image is resized to 256 by 256 pixels according to the image size set in step 1416. The resized image allows a Fast Fourier transform to be used to significantly reduce the time to calculate the Fourier spectrum.

At step 1418, the Fast Fourier transform is applied and the power spectrum is computed at step 1420. At step 1422, slopes of the Fourier spectrum at low and intermediate spatial frequencies are determined. The slope at low frequencies is determined by fitting a line over the region from the peak to where the spectrum has a value midway between its maximum and minimum values. The slope at intermediate frequencies is determined by fitting a line over the region between the spatial frequencies of 23 and 46 (pixel/2)⁻¹. The lower frequency region corresponds to spatial scales above fifty microns, and the intermediate frequency region corresponds to spatial scales between nineteen and thirty-eight microns.

At step 1424, a single parameter is derived from the Fourier spectrum. The parameter is the spectral ratio, S, which is the ratio of the slopes of the Fourier spectrum at low and intermediate spatial frequencies. The spectral ratio parameter is sensitive to the average size of vessels as well as the number of vessels in an image. Hence, at step 1426, S is evaluated to determine if it falls within a predetermined range. If the spectral ratio S lies within the interval between five and ten (i.e., 5<S<10), the image is considered to contain measurable vessels. Thus, at step 1428, the image is designated as passing the screening process.

Otherwise, at step 1430, the images are designated as failing. Images with S<5 generally contain predominately small vessels (e.g., diameters less than twenty microns) or few vessels. Images with S>10 generally contain large vessels (e.g., diameters greater than 100 microns) or highly uneven backgrounds with no discernable vessels. After the image as been screened, the control flow passes to step 128, as described above.

As is apparent from the foregoing description, the present invention was developed primarily to analyze blood components in a non-invasive manner. However, it will be clear to persons skilled in the relevant art(s) that the analysis techniques of this invention have utility beyond the medical applications described above. The invention has application outside the medical area and can be used generally to analyze visualizable components in a fluid flowing in any vascular system, such as a tube, the walls of which are transparent to transmitted and reflected light.

The present invention can be implemented using hardware, software or a combination thereof and can be implemented in one or more computer systems or other processing systems. In fact, in one embodiment, the invention is directed toward one or more computer systems capable of carrying out the functionality described herein. The present invention can be programmed in C, C++ and the like. In the preferred embodiment, C programming language is used.

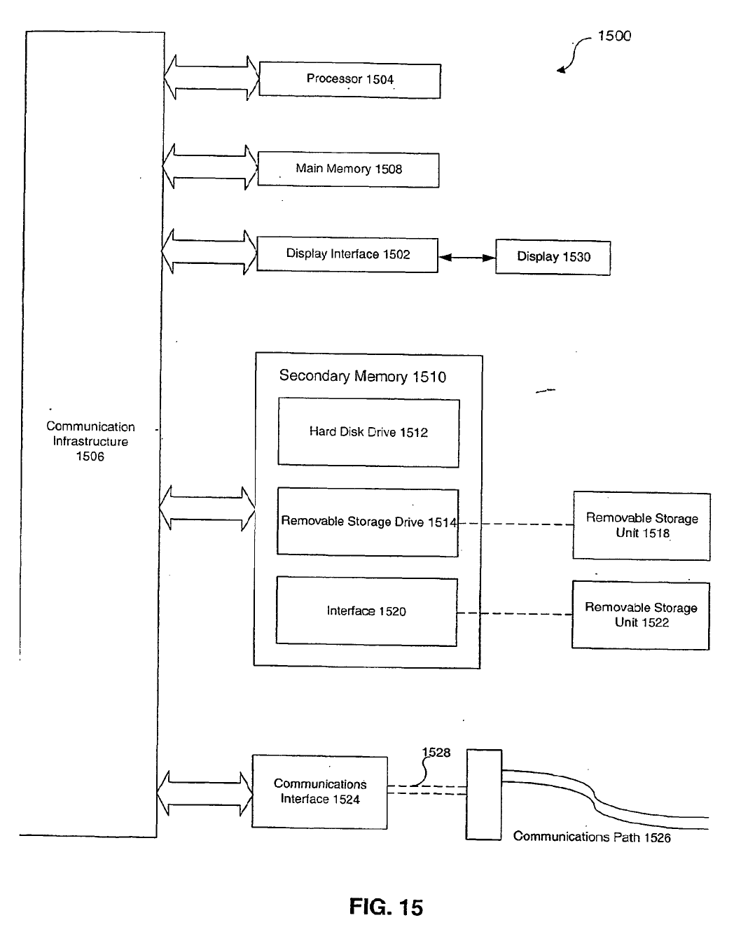

An exemplary screening, analyzing, and prediction means for use in the present invention is shown as computer system 1500 in FIG. 15. Computer system 1500 includes one or more processors, such as processor 1504. The processor 1504 is connected to a communication infrastructure 1506 (e.g., a communications bus, cross-over bar, or network). Various software embodiments are described in terms of this exemplary computer system. After reading this description, it will become apparent to a person skilled in the relevant art(s) how to implement the invention using other computer systems and/or computer architectures.

Computer system 1500 can include a display interface 1502 that forwards graphics, text, and other data from the communication infrastructure 1506 (or from a frame buffer not shown) for display on the display unit 1530.

Computer system 1500 also includes a main memory 1508, preferably random access memory (RAM), and can also include a secondary memory 1510. The secondary memory 1510 can include, for example, a hard disk drive 1512 and/or a removable storage drive 1514, representing a floppy disk drive, a magnetic tape drive, an optical disk drive, etc. The removable storage drive 1514 reads from and/or writes to a removable storage unit 1518 in a well-known manner. Removable storage unit 1518, represents a floppy disk, magnetic tape, optical disk, etc. which is read by and written to removable storage drive 1514. As will be appreciated, the removable storage unit 1518 includes a computer usable storage medium having stored therein computer software and/or data.

In alternative embodiments, secondary memory 1510 can include other similar means for allowing computer programs or other instructions to be loaded into computer system 1500. Such means can include, for example, a removable storage unit 1522 and an interface 1520. Examples of such can include a program cartridge and cartridge interface (such as that found in video game devices), a removable memory chip (such as an EPROM, or PROM) and associated socket, and other removable storage units 1522 and interfaces 1520 which allow software and data to be transferred from the removable storage unit 1522 to computer system 1500.

Computer system 1500 can also include a communications interface 1524. Communications interface 1524 allows software and data to be transferred between computer system 1500 and external devices. Examples of communications interface 1524 can include a modem, a network interface (such as an Ethernet card), a communications port, a PCMCIA slot and card, etc. Software and data transferred via communications interface 1524 are in the form of signals 1528 which can be electronic, electromagnetic, optical or other signals capable of being received by communications interface 1524. These signals 1528 are provided to communications interface 1524 via a communications path (i.e., channel) 1526. This channel 1526 carries signals 1528 and can be implemented using wire or cable, fiber optics, a phone line, a cellular phone link, an RF link and other communications channels.

In this document, the terms “computer program medium” and “computer usable medium” are used to generally refer to media such as removable storage drive 1514, a hard disk installed in hard disk drive 1512, and signals 1528. These computer program products are means for providing software to computer system 1500. The invention is directed to such computer program products.

Computer programs (also called computer control logic) are stored in main memory 1508 and/or secondary memory 1510. Computer programs can also be received via communications interface 1524. Such computer programs, when executed, enable the computer system 1500 to perform the features of the present invention as discussed herein. In particular, the computer programs, when executed, enable the processor 1504 to perform the features of the present invention. Accordingly, such computer programs represent controllers of the computer system 1500.

In an embodiment where the invention is implemented using software, the software can be stored in a computer program product and loaded into computer system 1500 using removable storage drive 1514, hard drive 1512 or communications interface 1524. The control logic (software), when executed by the processor 1504, causes the processor 1504 to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to persons skilled in the relevant art(s).

In yet another embodiment, the invention is implemented using a combination of both hardware and software.

While various embodiments of the present invention have been described above, it should be understood that they have been presented by way of example, and not limitation. It will be apparent to persons skilled in the relevant art(s) that various changes in form and detail can be made therein without departing from the spirit and scope of the invention. Thus, the present invention should not be limited by any of the above described exemplary embodiments.

What is claimed is:

1. A method for screening images of visualizable components in a fluid flowing in a vascular system, the walls of which are substantially transparent to transmitted and reflected light, using a light transmitting device that is capable of transmitting light through the walls of a region of the vascular system into a sub-surface region thereof, an image capturing device capable of capturing images reflected from the sub-surface region of the vascular system illuminated by the light transmitting device to create a spectral image, and a processing unit in communication with the image capturing device, comprising the steps of:
receiving in the processing unit a reflected spectral image of the subsurface region captured by the image capturing device;
screening said reflected spectral image to determine whether said reflected spectral image passes a screening stage, wherein said reflected spectral image passes said screening stage if at least one of a plurality of properties of said reflected spectral image exceeds a predetermined threshold; and
analyzing said reflected spectral image to measure an optical density of the vascular region to estimate hemoglobin, provided that said reflected spectral image passes said screening stage.

2. An method of claim 1, wherein said screening step further comprises:
uniformly extracting a predetermined number of rows and columns from said reflected spectral image to produce a subsampled image, wherein screening information is derived from said subsampled image for real time screening.

3. An method of claim 2, wherein said screening step further comprises:
evaluating said subsampled image to determine a mean image intensity value and a mean intensity variation value.

4. An method of claim 2, wherein said screening step further comprises:
performing DWT on said subsampled image to derive DWT coefficients for said rows and said columns at three decomposition scales.

5. An method of claim 2, wherein said screening step further comprises:
computing focus measurements from high frequency components of said subsampled image at a designated decomposition scale, wherein said focus measurements are used to compare focus conditions of said reflected spectral image and a controlled image.

6. An method of claim 5, wherein said focus measurements are automatically implemented to analyze an average vessel edge contrast of said reflected spectral image to determine if said reflected spectral image is out of focus.

7. An method of claim 5, wherein said screening step further comprises:
generating a binary vessel mask for said reflected spectral image, wherein said binary vessel mask includes a row mask and column mask.

8. An method of claim 7, wherein said screening step further comprises:
analyzing said focus measurements and said binary vessel mask for repetition detection to determine if said reflected spectral image and said controlled image are from the same site.

9. An method of claim 8, wherein said reflected spectral image passes said screening stage if said focus measurements of said reflected spectral image are higher than focus measurements of said controlled image.

10. An method of claim 1, wherein said screening step further comprises:
computing a motion blur measurement for said reflected spectral image.

11. An method of claim 10, wherein said screening step further comprises:
comparing said motion blur measurement to a motion blur threshold to detect motion blur, wherein said reflected spectral image does not pass said screening stage if motion blur is detected.

12. An method of claim 1, wherein said screening step further comprises:
performing vessel and bubble detection to determine the presence of good-sized vessels and bubbles in said reflected spectral image.

13. An method of claim 12, wherein the number of good-sized vessels and bubbles are compared to predetermined thresholds and said reflected spectral image does not pass said screening stage if said number of good-sized vessels or said number of bubbles exceeds said predetermined thresholds.

14. An method of claim 1, wherein said screening step further comprises:
cropping and subsampling said reflected spectral image according to a predetermined parameter to produce a subsampled image.

15. An method of claim 14, wherein said screening step further comprises:
applying a Fast Fourier transform to compute a power spectrum for said subsampled image.

16. An method of claim 15, wherein said screening step further comprises:
determining slopes of said power spectrum at low and intermediate spatial frequencies.

17. An method of claim 16, wherein said screening step further comprises:
deriving a spectral ratio from said slopes.

18. An method of claim 17, wherein said screening step further comprises:
evaluating said spectral ratio, wherein said reflected spectral image passes said screening stage if said spectral ratio falls within a predetermined range.

19. For use with a light transmitting device for transmitting light through a vascular system, and an image capturing device for capturing images from the vascular system illuminated by the light transmitting device to create a spectral image, a processing unit adapted for communication with the image capturing device for analyzing at least one visualizable component in a fluid flowing in the vascular system, the walls of which are substantially transparent to transmitted and reflected light, comprising:
receiving means for receiving a reflected spectral image of the vascular system captured by said image capturing device;
screening means for screening said reflected spectral image to determine whether said reflected spectral image passes a screening stage, wherein said reflected spectral image passes said screening stage if at least one of a plurality of properties of said reflected spectral image exceeds a predetermined threshold; and
analyzing means for measuring an optical density of the vascular system to estimate hemoglobin.

20. A computer program product comprising a computer useable medium having computer readable program code means embedded in said medium for causing an application program to execute on a computer that analyzes visualizable components in a fluid flowing in a vascular system, the walls of which are substantially transparent to transmitted and reflected light, using a light transmitting device that is capable of transmitting light through the vascular system, and an image capturing device capable of capturing images from the vascular system illuminated by the light transmitting device to create a spectral image, comprising:
a first computer readable program code means for causing the computer to receive a reflected spectral image of the vascular system captured by the image capturing device;
a second computer readable program code means for causing the computer to screen said reflected spectral image to determine whether said reflected spectral image passes a screening stage, wherein said reflected spectral image passes said screening stage if at least one of a plurality of properties of said reflected spectral image exceeds a predetermined threshold; and
a third computer readable program code means for causing the computer to measure an optical density of the vascular system to estimate hemoglobin, provided that said reflected spectral image passes said screening stage to determine whether at least one of a plurality of properties of said reflected spectral image exceeds a predetermined threshold.
